(19) United States
(12) Patent Application Publication
(10) Pub. No.: US 2007/0072187 A1
(43) Pub. Date: Mar. 29, 2007

(54) Novel methods and apparatus for cell based microarray assays

(76) Inventors: Herman Jacobus Blok, Retie (BE); Hendrik Sibolt Van Damme, Den Bosch (NL); Colin John Ingham, Wageningen (NL); Maria Helena Hilhorst, Wageningen (NL)

Correspondence Address: AMSTER, ROTHSTEIN & EBENSTEIN LLP, 90 PARK AVENUE, NEW YORK, NY, 10016, US

(21) Appl. No.: 10/579,896

(22) PCT Filed: Nov. 29, 2004
(86) PCT No.: PCT/EP04/13526

§ 371 Date: May 18, 2006

(30) Foreign Application Priority Data
Nov. 28, 2003 (EP) 03447276.1

(51) Int. Cl. C12Q 1/68; G01N 33/567; C12M 3/00; C40B 30/06; C40B 40/10

(52) U.S. Cl. 435/6; 435/287.2; 435/7.2

(57) ABSTRACT

The present invention relates methods for screening of cellular responses comprising: (a) providing a solid porous support having first and second surfaces and at least one area with a plurality of through-going channels (b) providing cellular components on said first and/or second surface of said solid porous support, wherein said solid porous support retains said cellular compounds on its surface; (c) providing a supply chamber at said first and/or second surface and opposite to said cellular components (d) subjecting all or part of said cellular components to one or more effectors; wherein at least one effector is delivered from said supply chamber through the porous support; (e) incubating the said all or part of cellular components with said effectors under conditions allowing the induction of cellular responses in the said all or part of cellular components; (f) optionally providing detector molecules to the said all or part of cellular components for assaying cellular responses (g) assaying for cellular responses; and, (h) identifying and characterizing the cellular responses induced by said effector molecules. The present invention further relates to the uses of said methods and apparatuses for carrying out said methods as well as to the use of a porous support for the preparation of a microarray kit for carrying out said methods.

### FIELD OF THE INVENTION

The present invention relates to the field of microarray technology. In particular, the present invention relates to delivery systems for delivery of effectors and/or reaction components within a microarray analysis system.

### BACKGROUND

In a range of technology-based business sectors, including the chemical, bioscience, biomedical, and pharmaceutical industries, it remains increasingly desirable to develop capabilities for rapidly and reliably carrying out chemical and biochemical reactions in large numbers using small quantities of samples and reagents.

There has been a growing interest in the development and manufacturing of microscale fluid systems for the acquisition of chemical and biochemical information and as a result of this effort, microfluidics is considered an enabling technology for providing low cost, high versatility devices to operations, such as drug lead discovery technologies.

Microfluidic devices as currently in practice include typical two-dimensional devices where often DNA probes are tethered to flat surfaces. Limitations to such 2D devices, including the limited detection limit by the quantity of DNA that can be bound to a two dimensional area and the rate-limiting step introduced by such a flat surface, however, have led to efforts to increase the analysis efficiency resulting in the development of three-dimensional devices such as disclosed e.g. in EP 0 975 427 and U.S. Pat. No. 6,383,748 B1. These 3D devices comprising a porous structure allow the tethering of probes within densely packed pores or channels and allow so-called flow-through analysis whereby a sample to be analyzed can be flown through said channels for efficient reaction or hybridization to the tethered probes.

High-throughput 3D microarray technology has greatly improved the efficiency of chemical and biochemical analysis, synthesis and screening procedures. With the advent of combinatorial chemistry approaches to identify pharmacologically useful compounds, it is increasingly evident that there is a need for methods and apparatuses at microarray levels, capable of performing high-throughput characterization of pharmacological profiles and corresponding potencies of the compounds in the synthesized combinatorial libraries.

Living-cell-microarray technology provides a short-cut to the development of safer and more customized personal drugs and a better understanding of the molecular pathways in the functioning of cellular organisms. Microarrays of living cells and methods for high-throughput screening of cellular responses of cells or cellular components were developed by PamGene B. V. as disclosed in International Application PCT/EP03/05798.

As the new-generation cellular assays are more complex and demanding, a need is created towards multiplex microarray analysis of various targets within a single cell, offering researchers a closer look at living systems in a high-throughput manner.

As will be well appreciated in the art, there is a continuous need for improved methods and apparatuses for cell-based assays.

It is therefore an object of the present invention to provide novel devices and methods for high-throughput microarray analysis for cell-based assays; easily accommodating a high level of analysis complexity.

### SUMMARY OF THE INVENTION

High content cellular screening in whole living cells allows researchers to observe the effects of compound-target interaction, determine toxicity and specificity of compounds, and identify cell-to-cell variability in drug response. It also allows researchers to screen targets that are intractable using conventional in vitro assays. Availability of high-content information in primary screening promises to increase confidence in hits and reduce the need for secondary screens.

Availability of high-content cellular information at early stages of drug discovery promises to improve the quality of targets, hits, and leads; reduce late-stage attrition; and shorten time and cost of development.

The present invention allows multiplex analysis of compound interactions with cells or cellular components whereby exposure of said cells or cellular components to (a) one or more compounds, (b) compound concentration and (c) one or more compounds in function of time can be varied in a parallel manner.

The present invention thus provides a highly multiplex analysis method for screening of cellular responses comprising:
(a) providing a solid porous support having first and second surfaces and at least one area with a plurality of through-going channels;
(b) providing cellular components on said first and/or second surface of said solid porous support, wherein said solid porous support retains said cellular compounds on its surface;
(c) providing a supply chamber at said first and/or second surface and opposite to said cellular components;
(d) subjecting all or part of said cellular components to one or more effectors; wherein at least one effector is delivered from said supply chamber through the porous support;
(e) incubating the said all or part of cellular components with said effectors under conditions allowing the induction of cellular responses in the said all or part of cellular components;
(f) optionally providing detector molecules to the said all or part of cellular components for assaying cellular responses
(g) assaying for cellular responses; and,
(h) identifying and characterizing the cellular responses induced by said effector molecules.

In addition to its ability to perform highly efficient multiplex analysis on a microarray platform, the present invention additionally allows the delivery of compounds to arrayed matter that otherwise with traditional techniques would suffer undesirable effects.

In current practice, typical techniques for delivery of reaction components onto microarrayed biological or bio-molecular material include spotting or printing of said reaction components through an array of tweezers, pins or capillaries that serve to transfer or deliver any content within the delivery mechanism to the surface by either physically tapping said tweezers, pin(s) or capillary(ies) on the surface or by spraying.

Although proven to satisfy most applications, current spotting or printing techniques may suffer shortcomings towards some reactants as they may clog the spotter by forming aggregates. Also, some compounds spot poorly due to charge or unknown contaminants that cause the spots to change path during flight and therefore spot in the wrong location. Viscosity or chemical reactivity with spotter components may further cause unwanted difficulties while spotting.

Once spotted, tethered reactants or compounds may suffer loss of reactivity due to the dried format in which often printed microarrays are stored and/or sold. Some reactants or compounds may not re-hydrate properly as can be expected from hydrophobic compounds including lipids. For example, in terms of activity, it is for some enzymes very hard to maintain their activity and they may irreversibly denature even if freeze-dried. In addition, timed addition and removal or changes in the concentration of a spotted compound during an assay is hard to achieve with current technology.

The present invention overcomes the aforementioned disadvantages in addition to high-throughput multiplex analysis that allows increased data acquisition in a single experiment.

In addition to the aforementioned advantages, devices according to the present invention may avoid incompatibilities due to the solvent which accompanies a reactant with an envisaged assay. I.e. the solvent usually used to dispense a reactant(s) may be removed by drying and said reactant(s) stored within the device according to the present invention after which contact with an appropriate liquid or buffer then allows participation of said reactant(s) in said assay, thereby avoiding possible interference of said solvent with the subsequent assay.

Devices according to the present invention further allow efficient filtration steps through the porous support when harvesting cells. Removal or replacement of media while retaining the cells may be simply by placing of the porous support in a suction device (such devices are known for 96 well filter plates); this step would otherwise need time-consuming centrifugation and removal of liquid by pipetting.

The present invention further discloses uses of the above method according to the invention.

Additional features and advantages of the invention will be set forth in the detailed description which follows, and in part will be apparent from the description, or may be learned by practice of the invention. The objectives and other advantages of the invention will be realized and attained by the process particularly pointed out in the written description and appended claims.

### DETAILED DESCRIPTION OF THE INVENTION

Before the present methods and devices of the invention are described, it is to be understood that this invention is not limited to particular methods, components, or devices described, as such methods, components, and devices may, of course, vary. It is also to be understood that the terminology used herein is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein may be used in the practice or testing of the present invention, the preferred methods and materials are now described.

In this specification and the appended claims, the singular forms “a”, “an”, and “the” include plural references unless the context clearly dictates otherwise.

The present invention provides a system for high-throughput screening that is automation-friendly and allows parallel processing of numerous tests. Devices according to the present invention comprise a plate or carrier with an array of test areas arranged in rows and columns, wherein the bottom of each test area is a solid porous support having first and second surfaces and at least one area with a plurality of through-going channels. Each porous solid support in a test area or well may comprise a microarray. The present invention therefore relates in particular to an array of arrays. It is understood by the term “test area” or “well” that these represent areas of the array which direct test compounds or other reactants or cellular components or samples onto the solid support(s). Said areas may have a depth or a height or may be planar with respect to said plate or carrier in which the individual arrays are hold. Said test areas may further have any suitable shape including without limitation circular shape, square shape, rectangular shape and the like.

The present invention provides for a multiplex microarray analysis of responses of cellular components or cells to effectors. Effectors and effector molecules, cellular components and optionally detector molecules and capture molecules may be introduced on the solid porous support in a multiplex way. In particular, the provision within the present invention of a supply chamber provides for an additional dimension allowing parallel delivery of one or more reactants towards both first and second surfaces of the solid support.

The term “reactant” as used within the present specification refers to any component or treatment provided to the solid support in order to perform the methods according to the present invention, i.e. cellular components, effector, effector molecules, detector molecules and capture molecules. An effector molecule may be any molecule which may induce a cellular effect. It is understood within the meaning of the present invention that both terms “effector” and “effector molecule” may be included in the general common term “effectors”. An effector is a variable component in the assay and not a common component of the array environment, i.e. not a universal component of the growth medium.

Supply Chamber

As will be well appreciated, a supply chamber as provided within the present invention allows the delivery of reactants to the solid support which otherwise may suffer impracticalities; e.g. which may clog the capillaries of e.g. a spotting device.

Depending on the assay which is envisaged, a supply chamber according to the present invention may be positioned towards the first or the second surface of the solid support, corresponding to positions respectively along and opposite to the outer first surface onto which the cellular components are deposited. Said position along the outer surface onto which the cellular components are deposited provides for a direct contact of said cellular components and the reactants delivered by the supply chamber; i.e. said reactants are not transferred through the porous solid support prior to contact with said cellular components. Alternatively, two supply chambers may be provided adapted to receive the solid support sandwiched in the interface between the supply chambers. The present invention further contemplates the provision of cellular components to both first and second solid support surfaces sandwiched in the interface between two supply chambers. Particular useful devices according to the present invention comprise cellular components on the first or second surface of the solid support and a supply chamber in contact to the surface of the solid support that is opposite to the surface provided with cellular components.

A supply chamber as provided with the present invention gives access of its content to at least one array within an array of arrays (FIGS. 1A and B) to which it is attached by either mechanical attachment (e.g. click on system or other), physical attachment or merely by being in liquid contact with the array. Physical attachment of the supply chamber to the solid support may be, by way of example and not limitation, thermal bonding, laser welding, ultrasonic welding, latex masking agents, glues or chemical welding (chemical solvent-based bonding). A washing step usually follows to remove any possible toxic product that may be derived from the attachment procedure. Said physical and/or liquid contact may not be permanent and as such allows subsequent supply chambers with diverse or equal contents to be combined with a same solid porous support. A removable supply chamber according to the invention offers the advantage and flexibility of transferring effectors to the cellular components on the solid support and immediate interruption of said supply by removal of the chamber.

Accordingly, in one embodiment of the present invention, methods are provided, wherein said supply chamber is in liquid contact with said first and/or said second surface of said solid support.

Liquid contact may be simply by orienting a supply chamber to the surface of the solid support that is opposite to the surface carrying cellular components and optionally orienting the whole so as to achieve a downwards liquid transfer of the content within the compartments (e.g. a liquid medium or an agent to modify flow rate such as a gel or detergent) to the solid support underneath. It is noted that said orienting the whole so as to achieve a positioning of the solid support underneath the supply chamber may not be necessary as the capillaries within said solid support may draw the liquid into them and this may be upward as well as downward). Alternatively, the solid support may simply rest on a liquid reservoir such as a dialysis membrane filled with liquid.

Non-limiting examples of supply chambers that may be in liquid contact with a solid porous support according to the present invention include gel patches and open capillaries that contact the porous solid support.

Physical attachment may be by resting the solid support on a solid matrix such as a gel or other porous support from which fluid is drawn. Physical attachment may provide structural support to the device.

Both liquid contact and solid attachment does not exclude the solid support as being part of the structure of the device in its entirety.

A supply chamber according to the present invention comprises a planar square, rectangular or circular surface and four upstanding walls surrounding the circumference of said surface to form a chamber having an open top and a closed bottom surface. The open (top) end of the supply chamber is oriented towards the first or the second surface of the solid porous support to which it becomes then physically attached or by liquid contact with the array. Useful supply chambers may also have open top and bottom surfaces.

The present invention thus also contemplates a device for performing a method according to the present invention, comprising a solid porous support; said support being at its first and/or second surface in liquid contact with a supply chamber or in gaseous contact or wherein said supply chamber may be physically attached thereto; wherein said supply chamber comprises multiple-use insertions, said multiple-use insertions are fixed or movable separations and wherein the spatial organization of the inserts determines the number of compartments.

In fact, the structure of the supply chamber may be in physical contact with the solid support; however; in particular the porous support draws liquid into the capillaries or pores, even if the liquid comes from a gel (e.g. agar) permeated with e.g. nutrients and other compounds—as such liquid contact is critical. Alternatively, a supply chamber according to the present invention may be attached to the porous solid support by gaseous contact; e.g. biogas sniffers.

A supply chamber according to the present invention may comprise multiple-use-insertions for parallel studies (FIGS. 1A and B). Multiple-use-insertions are fixed, or optionally movable, separations allowing the supply chamber to be compartmentalized. The spatial organization of the inserts determines the number of compartments and the number of arrays covered by one compartment (FIG. 1B). If no inserts are used, the supply chamber is likely to comprise one compartment.

A supply chamber comprising no movable insert and hence a single compartment is particularly useful when a single effector or a single mixture of effectors or a gradient of one or more effectors is to be supplied towards the porous solid support. Two-dimensional gradients in particular offer to each position on the porous solid support a unique environment. Alternatively, multiple compartments may be present each with their own gradient of effectors.

Accordingly, in one embodiment of the present invention, methods are provided wherein said supply chamber comprises at least 1 compartment; i.e. 2, 3, 4, 5, 6, 7, 8, 9, 10 or even more compartments.

The number of compartments may be limited to the number of spots or pre-defined regions printed on the solid support. However, larger pre-defined regions may be served by more than one compartment. The number of compartments in a supply chamber may also be limited according to the manufacturing of the device.

In another embodiment, a supply chamber as described herein is provided, wherein said at least one compartment is provided with one or more effectors for performing a method according to the present invention.

In a further embodiment, a supply chamber is provided, wherein said at least one or more effectors is contained within a gaseous or liquid medium.

Use of a Supply Chamber for Performing Methods According to the Present Invention

In a further embodiment of the present invention, a device is provided comprising a solid porous support and thereto attached a supply chamber, wherein said supply chamber comprises at least one compartment.

The at least one effector or effector molecule transported towards the porous solid support via the supply chamber may be contained within a solid, liquid or gaseous medium depending on the nature of the effector. For example, nutrients to induce and/or maintain growth of cells inoculated on an outer first surface of the porous solid support will usually be contained in a growth medium and provided from a supply chamber oriented with the open end towards the opposite outer second surface. Growth medium is typically provided as a liquid or gel medium including e.g. nutrient broths and agar or agarose gel containing nutrients. Typical cell growth medium may be any conventional medium suitable for growing cells, such as minimal or complex media. Suitable media are available from commercial suppliers or may be prepared according to published recipes (e.g. in catalogues of the American Type Culture Collection). The media are prepared using procedures known in the art.

Accordingly, in one embodiment of the present invention, methods are provided, wherein said at least one compartment within the supply chamber is provided with a liquid medium comprising at least one effector molecule.

In a further embodiment, methods according to the present invention are provided, wherein said at least one compartment is provided with a liquid medium comprising a gradient of at least one effector molecule.

In yet a further embodiment of the present invention, methods are provided wherein said at least one compartment is provided with a liquid medium comprising a 2D gradient of at least two effector molecules, e.g. 3, 4, 5, 6, 7, 8, 9, 10 or more effector molecules.

2D gradients in more than one compartment of a supply chamber may comprise an equal composition of effector molecules, said effector molecules in each compartment may differ or not in concentration.

The complexity may depend on the nature of the medium, i.e. for example a serum may present a very complex mixture of effectors. A mixture of effectors may also be accomplished by manual preparation; in this case the amount of each effector in said mixture is exactly known.

A supply chamber according to the present invention may comprise fixed inserts to form a supply chamber with a fixed number of spatially arranged compartments.

The reversibility of supply chamber attachment allows removal of a nutrient layer that may interfere with an assay due to for example auto-fluorescence or other issues related to detection or preparation of cells for storage and/or archiving. The removable supply chamber also permits sequential addition of effectors or gradients of effectors.

If a number of porous solid supports in the array of arrays need to be excluded from delivery of effectors via the supply chamber, than this may be achieved simply by leaving the corresponding compartments empty or by blocking them for any material transfer.

The supply chamber according to the present invention may be manufactured from materials as well known in the art and suitable for receiving and storing of biological material such as metals including stainless steel and alloys, glass and plastics polymers. These materials preferable have a good chemical resistance, have stable physical properties, may be rigid, semi rigid or flexible and may exhibit any degree of translucence or opaqueness depending on the material stored within the supply chamber. Any materials that can be coated or chemically modified are suitable as well. Suitable materials are further preferably anti-fluorescent and do not allow the volume on the compartment(s) to change during the analysis. Plastics are particularly suitable materials for the manufacture of supply chambers according to the invention and may include natural polymers such as e.g. latex as well as chemically modified polymers such as e.g. vulcanized rubber and bakelite. Non-limiting examples of plastics for manufacture of supply chambers according to the invention include polyethylene terephthalate (PET, PETE), high density polyethylene (HDPE), polyvinyl chloride or PVC, low density polyethylene (LDPE), polypropylene, polystyrene, liquid crystal polymers (LCP), Topas® including combinations thereof.

Multiplexity of analysis provided by the methods of the present invention is at multiple levels including (a) supply of reactants at first and/or second surface of the solid support, (b) positionally directed supply to one or more arrays of at least one reactant from the supply chamber, and (c) provision and storage of effectors or other reactants within the porous structure of the solid support prior to assay performance.

According to the methods of the present invention, a supply chamber as described within the present specification allows access to the solid porous support of effectors or other reactants by either diffusion or active transfer.

Liquid contact of the supply chamber with the solid porous support allows diffusion of the effectors from the supply chamber through the porous solid support. Further, effectors may be passively transported by capillary action, by osmotic action, by liquid contact force or by convection. The term “contact force” as used within this specification means a direct surface contact between the solid porous support and the means for delivery of effectors or other reactants such as a supply chamber. Surface contact related to the supply chamber may be by the liquid surface of the medium within the chamber.

Active transfer of effectors from a supply chamber may be for example by pumping (both pushing and drawing), acoustic wave, by application of a low pressure above the solid support, or by vapour contact.

Accordingly, in one embodiment of the present invention, methods are provided, wherein the said at least one effector molecule is transported passively or actively through said porous support.

In a further embodiment of the invention, methods are provided, wherein the said at least one effector molecule diffuses through said porous support to the cellular components by contact force.

Alternatively, diffusion of effectors or other reactants through the pores of the solid porous support may be an active diffusion by for example active pumping, magnetic force, electrical force or piezo-electric force.

Accordingly, in a further embodiment of the invention, methods are provided, wherein the said at least one effector molecule is transported actively through said porous support by pumping, magnetically, electrically, or by piezoelectric force.

According to the position of the open end of the supply chamber relative to the first or second surface of the solid porous support and according to the general orientation of the combination of both supply chamber and solid support in space, the supplied reactants will diffuse from the supply chamber upwards or downwards through said solid support.

Hence, it is another object of the present invention to provide a supply chamber for spatial delivery of one or more effectors through a porous solid support comprising:
(a) multiple-use insertions, said multiple-use-insertions are fixed or movable separations and wherein the spatial organization of the inserts determines the number of compartments, said supply chamber comprising at least one compartment, said at least one compartment allowing said delivery of one or more effectors through part or all of the channels within said porous solid support;
(b) means for compartment alignment towards predefined regions on the support;
(c) means of adding or removing or changing the amounts of effectors.
   Delivery of Reactants to the Support by Other Means

In addition to effector supply to the porous solid support via the supply chamber, delivery of additional effectors and other reactants that may be provided by other means may be provided via spotting. Spotting of effectors may be preferred in case an effector would for example be insoluble or too large to diffuse through the pores of the support. Sometimes, spotted compounds may show more stability during storage or during assaying.

Delivery of effectors, cellular components or detector molecules to predefined regions on the support may be accomplished by using a liquid handling device but may equally be accomplished by manual handling. Examples of defined areas of the array include different XY positions on a planar porous support and may also take account of other forms of localization, such as effectors localized predominately on the upper or lower surface of the support or within individual pores.

Accordingly, a liquid handling device may be positioned on the solid support, wherein said liquid handling device may be a high precision x-y-z pipettor or inkjet printer containing 1 or more channels through which liquid can be dispensed, sequentially or in parallel, to positions corresponding to arrayed molecules on the surface of the solid support. Alternatively, a superposing mask comprising transversal holes may be superposed onto the support, wherein said superposing is such that each transversal hole in said mask corresponds to a predefined region on the surface of said solid support.

Superposing masks may be useful in the generation of cellular arrays. In general, a mask delineates areas on the solid support onto which cells may grow and/or onto which molecules or compounds may be spotted/immobilized. After growing a confluent layer of cells, a mask may be used for said cells to become subsequently transformed by directing a set of vectors or gene-constructs to predefined areas on the confluent layer of cells so as to obtain an array of different transformed cells.

The use of a mask during the transformation step allows the transformation of cells growing on a predefined area on the array to be transformed with a known vector or gene-construct. As such, through the use of a mask, an XY-paftern of transformed cells is created, of which the XY-position on the array identifies the transformed cells.

Further an array of living cells may be obtained by dropping molten agar spots onto the porous solid support. The porous nature of the support draws the molten agar into the pores by capillary action. The agar-spotted support may then subsequently be overlaid with cells that will only grow at predefined regions on the support determined by the positions of the agar spots. Alternatively, suitable gels or polymers, possibly interconvertable from fluid to gel by methods other than temperature shifts or also by changes in temperature, may also be used in place of agar in the present invention.

Suitable superposing masks are made of inert material and prevent microbial cross-contamination. Particular useful masks are penetrative and compartmentalize the porous solid support.

Accordingly, in one embodiment of the present invention, a device as described herein is provided comprising a solid porous support and a supply chamber, wherein an array of cellular components is provided in predefined regions on the surface of said support.

In a further embodiment, a device as described herein is provided comprising a solid porous support and a supply chamber, wherein said cellular components are conditioned for preservation on said support.

In yet a further embodiment, a device as described herein is provided comprising a solid porous support and a supply chamber, wherein said cellular components are conditioned for preservation on said support and wherein said condition is chosen from the group comprising lyophilization, liquid nitrogen and glycerol dissolution.

Some assays may require a continuous layer of cells over the whole or part of the first and-or second surface of the porous solid support rather than an array of cells.

Accordingly, in one embodiment, a device as described herein is provided comprising a solid porous support and a supply chamber, wherein a cellular component is provided on the surface of said support.

In a further embodiment, a device as described herein is provided comprising a solid porous support and a supply chamber, wherein a cellular component is provided on the surface of said support, said cellular component being conditioned for preservation on said support.

Delivering of effectors, cellular components or detector molecules may be by means of contact or non-contact spotting. The term “contact spotting” or “contact force” as used in this specification means a direct surface contact between a printing substrate and a delivery mechanism that may contain one or a plurality or an array of tweezers, pins or capillaries that serve to transfer or deliver any content within the delivery mechanism to the surface by physically tapping said tweezer(s), pin(s) or capillary(ies) on the surface. Further, a superposing mask may be positioned on the (cells-containing) solid support whereby the content of the wells as formed by the filled holes in the mask is passively delivered onto said cells by capillary actions when pressing the mask onto the chip. As used in the present specification, a mask acts as a barrier to the passage of a reagent. Typically, a pattern of holes in the mask allows selective passage of reagent and results in a corresponding pattern of reagent deposition on a surface placed behind/below the mask.

Alternatively, the effectors may also be delivered or spotted through ink-jet printing technology, a non-contact technology in which reactants are sprayed onto the surface using technology adapted from computer ink-jet printers. The ink-jet method is sometimes called indirect because the reactants are sprayed onto the surface rather than being directly placed. Ink-jet methods may be capable of producing smaller spots, and because they avoid physical contact with the surface may prove to be more reliable.

Useful ink-jet printing methodologies may include continuous and drop-on-demand ink-jet methods. Most suitable ink-jet printing methods are drop-on-demand ink-jet methods, examples of which include piezoelectric and electrostatic ink-jet systems.

Further useful, in the present invention are spotting robots or liquid handling devices. Most spotting robots or liquid handling devices use an X-Y-Z robot arm (one that can move in three dimensions) mounted on an anti-vibration table. Said arm may hold nozzles in case of non-contact spotting. In contact spotting, said arm may hold pins. Nozzles or pins are dipped into a first microtiter plate to pick up the fluid to be delivered. The tips in case of pins are then moved to the solid support surface and allowed to touch the surface only minimally; the fluid is then transferred. The pins are then washed and moved to the next set of wells and fluid. This process is repeated until hundreds or thousands of compounds or molecules are deposited. Solid pins, quills, and pin- and- ring configurations of pins may be useful.

Accordingly, in one embodiment of the present invention, delivery of at least one effector is from above the support by a means chosen from the group comprising a delivery mask, a microfluidics device, a high precision x-y-z micro-pipettor, inkjet printer, and manual handling.

Further, delivery of effectors by means other than a supply chamber to the cells-containing support may be by means of a contact force which may be a capillary force or a piezo-electric force.

Alternatively, transfer of e.g. effector/detector molecules to cellular components on the solid support may also be by providing said effector/detector molecules to a first solid support which is then placed on a second solid support carrying the cellular components. The effector/detector molecules are subsequently transferred onto the cells (in an arrayed layout or not) by e.g centrifugation or suction

As will be well appreciated in the art, the combination of supply chamber and liquid handling devices or microfluidics devices allow high-multiplexed cell-based analysis of a broad variety of effectors and/or other reactants. Microfluidics devices may also be attached to a supply chamber; e.g. a solid metal block having channels going through it wherein each channel can address a whole or part of a solid support—the reservoir for fluid delivery to the solid support may be outside the supply chamber.

Compound Screening

The use of compound libraries is particularly known to speed up drug discovery. Precipitation of some compounds is a recognized problem and known to occur with a large number of potent lead compounds. Due to the precipitation, often these compounds are excluded from screening programs because of the otherwise clogging of the liquid handling systems. A solution to this problem is provided by using a supply chamber according to the present invention. Large compound libraries may be stored within a multiplicity of supply chamber compartments, ready for use in a cell-based assay.

Compound libraries may be stored in the supply chamber. They may be present in dry condition after e.g. slow evaporation or vacuum drying methods or e.g. by blowing air above and below the wells. Dried compounds can be dissolved later on when an assay needs to be performed. Alternatively, said compounds may be in solution already. Depending on the solubility of the compound, diffusion may be total or partial and sufficient to allow for hit identification. Transfer of the compounds is not limited to diffusion, and may also be by pulsing a liquid sample back and forth through the porous support thereby maximizing mixing of assay components. By pulsing a sample within the pores of the support, compounds in the supply chamber may be pulsed along.

Alternatively, compounds useful in the discovery process of drug candidates may be provided and stored within the porous structure of the solid support. Devices according to the present invention comprise a plate with an array of wells arranged in rows and columns, wherein the bottom of each well is a solid porous support with a plurality of through-going channels. Compounds may be dispensed into each of the wells and dried or concentrated into the porous support using e.g. slow evaporation or vacuum drying methods or by e.g. by blowing air or an inert gas such as e.g. helium above and below the wells. These library plates may be stored until assay performance. Assays are directly performed in these compound plates by adding the appropriate buffers and further essential components. The use of these compound plates avoids laborious and time consuming compound distribution. A sample is pumped up and down within the pores of the solid support and measurements are by fluorescence, chemiluminescence or radiometric imaging.

Accordingly, in a further embodiment of the present invention, an effector is a drug or any compound which is useful in the discovery process of a drug candidate.

In yet a further embodiment, said effector is a drug selected from a chemical or natural drug candidate library.

Accordingly, the present invention contemplates the use of compound plates as described within the present specification enabling a further increase of the multiplex character of the present invention.

Optionally, compound plates as disclosed herein may comprise a coating to affect slow or controlled drug release into the assay medium once the plate or the porous solid support is provided with buffer at the initiation of an assay. Such a coating finds particular use if a timely dosage of drug into the assay medium is required over a longer period of time (e.g. with screening of C. elegans or any other cellular screen).

Accordingly, in another object of the present invention to provide a solid porous support, wherein within its porous structure an array of test compounds is provided in dried, lyophilized, gaseous or supercritical state

Accordingly, in one embodiment, a device according to the present invention is provided comprising a porous solid support and a supply chamber, wherein an array of test compounds is provided within predefined regions on the surface of said support, said test compounds are in liquid, gaseous or supercritical state.

Said test compounds are usually not immobilized within said porous support. However, test compounds may be immobilized temporarily e.g. with triggered release (e.g. temperature, or laser activated release) or e.g. whilst still immobilized may have an effect on a cellular component e.g. through surface interactions. Alternatively, compounds may be immobilized temporarily with a release that is susceptible to a specific cleaving agent either chemical or enzymatic such as e.g., a nucleic acid sequence that contains the recognition site for a restriction endonuclease, or a specific peptide (or protein) that contains the cleavage site for the corresponding peptidase (or protease).

Test compounds may be immobilized within the porous structure of the solid support temporarily (e.g. to provide a defined release rate) or permanently wherein the permanently immobilized compounds may still have an effect on a cellular component e.g. via external receptors. Test compounds may also be immobilised within the supply chamber from where they may be delivered to the cellular components after having first entered a gas or liquid phase.

Reactants

In general, there are a number of reactants involved in the cellular arrays according to the present invention including cellular components and one or more effectors and optionally also detector molecules. In addition, also cell-capturing molecules may be involved; these may be for example antibodies, lectins or aptamers to capture a specific bacterium each. Depending on the nature of the capture molecules, specific cells (bacteria, fungi, viruses, mycoplasmas, mammalian cells) may be captured. A variety of distinct capture molecules on an array may provide for a cellular array comprising a variety of distinct cellular components. The present invention provides a versatile integrated cellular-based assay wherein a number of test formats are envisaged.

In an array of cellular components, islands of different cells are grown or deposited on the support in an array format. Subsequently the whole array is exposed to one or more effectors and finally exposed to one or more detector molecules (possibly present in the substrate) if necessary after lysis. As such, this test format allows the screening of an array of different cellular components for responses induced by at least one particular effector, detected with a particular detector molecule. Said detector molecule(s) may be provided subsequent to the incubation of the at least one effector with the cellular components or may have been introduced within the support prior to contact of the support with the cellular components. In addition, a detector molecule may have been introduced into the cellular components prior to exposure to the effectors; e.g. GFP may be expressed as a cellular response.

Cellular components may be captured on the solid support by capture molecules which were previously deposited onto said solid support.

The term “detector molecule” refers, in the context of the present invention, to molecules which allow the detection of a cellular response. A detector molecule may also be generated by the conversion of an effector.

In an effector array a homogeneous layer of a cellular component is locally, at predefined regions, treated with at least one effector. The at least one effector may be present (a) in the substrate before the cellular components are applied, (b) in the cells, (c) may be spotted from the top of the support onto the layer of cellular components or (d) may be delivered to the cellular components from a supply chamber which is in fluid contact with the array support. After treatment, cellular responses may be detected with a particular detector molecule. Said detector molecule may be provided subsequent to the incubation of the effector with the cellular components or may have been introduced within the porous solid support prior to contact of the substrate with the cellular components. Also, the detector molecule may have been introduced in the cells such as for example to obtain GFP-expressing cells.

In a detector array, an array of different detector molecules is contacted with a homogeneous layer of cellular components which are treated with at least one particular effector. Cellular responses are monitored by detecting excretion products by the detector molecules or by detecting intracellular products through binding to the receptor molecules, optionally after lysis of the cellular components. Cell death and morphological changes may also be detected.

Living cells typically require control of such factors as temperature, pH, and humidity in order to maintain viability. Furthermore; the cells must be protected from contamination of external agents such as bacteria. In some cases, it is necessary to protect laboratory personnel from contamination by the cells (i.e. viral cell lines and pathogenic microorganisms). If high-sensitivity fluorescence detection is being used, then dust particle contamination must be kept to a minimum, as dust causes false positive readings for these kinds of detection systems

In order to prevent contamination the supports and devices according to the present invention may be closed off. Alternatively, devices according to the present invention may be enclosed within a controlled environmental chamber. There are various options available depending on the specific requirements for protection of the samples and the laboratory personnel. Laminar flow hoods provide a protective air curtain along with positive pressure to protect the inside contents from external contamination, such as from airborne bacteria. These, however, do not protect personnel in the lab. Biosafety cabinets incorporate a combination of airflow control and HEPA filtration to protect both the contents of the cabinet and the people outside. There are several types of Biosafety cabinets as known in the art and specified by the CDC (Centers for Disease Control).

Support and supply chamber may also be enclosed in an integrated small encapsulating device that retains pathogens within the device, obviating the need for working in a laminar flow or Biosafety cabinet once the pathogens have been transferred onto the support.

Cellular Components

The term “cellular components” as used throughout the present specification refers to whole intact viable cells including, e.g. prokaryotic and eukaryotic cells; as well as cell components such as vesicles, organelles, part or whole of cell content(s), and vectors; as well as sectioned material such as tissue sections; as well as fixed cells; as well as microscopic multicellular organisms such as, e.g., nematodes and others. Cellular components may be also bacteria and mycoplasmas and agents infective to cells such as viruses where the potential exists for the virus to interact with cells on the array at some point in the assay.

According to the present invention, the surface of said solid support may be contacted, by direct deposit thereon, with an inoculum of cellular components. Said inoculum may be a liquid formulation comprising said components and an appropriate growth medium; usually in concentrated form and small volume quantities. An inoculum may eventually be introduced on the support in a diluted form.

The final inoculum, however, may also be disposed of any growth medium and comprise preservers instead such as glycerol (e.g. bacterial cultures). Accordingly, cellular components may be preserved on the substrate for analysis later on; i.e. cellular components may be on the substrate under preserving conditions such as in glycerol or other suitable medium or lyophilised. The term “preserving condition” refers to a condition to keep the cellular components alive and/or intact and free from decay.

Alternatively, cellular components may be cultivated for growth until the exponential phase with respect to their growth curve is reached corresponding to an indicative optical density, followed by deposition of an aliquot of said culture directly on the substrate.

Cellular components or structures may be equally provided in the general form of a solution or physiological solution, e.g. when providing microsomes, ribosomes, endoplasmic reticulum, mitochondria or mitochondrial cristae and other cellular vesicles. The present invention also contemplates the use of mixtures of cultures or inoculum mixtures and mixtures of the above-mentioned solutions or any mixture thereof.

Accordingly, in one embodiment of the present invention a method is provided wherein said providing of cellular components on the surface of a substrate is by a deposit directly on said substrate of an inoculum, culture, solution, or mixtures thereof. Deposition of mixtures of an inoculum, culture or solution may be simultaneous or sequentially.

As will be appreciated by a person skilled in the art, established protocols are available for the culture of diverse cell types and the isolation of cell structures or cell vesicles. Such protocols may require the use of specialized coatings and selective media to enable cell growth and the expression of specialist cellular functions. None of such protocols is precluded from use with the method of the present invention.

In the present invention, nutrients may be provided to the porous solid support from underneath or from above and through the pores of said solid support. According to the present invention, nutrients are in particular supplied via the supply chamber which may be oriented with its open end towards the first and/or second surface of the solid support. Usually, a nutrient supply chamber is placed to the outer surface of the solid support which is opposite to the surface on which the cellular components are introduced.

Besides nutrients, one or two additional effectors may be included in the supply chamber if parallel transfer is required. Alternatively, additional supply of effectors such as for example agonists and antagonists will usually be via a second supply chamber or by use of a compound plate as described within present specification

The methods according to the present invention may also be applicable to sectioned material which may be directly positioned in contact with the support.

If required for downstream assays, e.g. immuno-fluorescent detection, cells or cellular components may be fixed and/or permeabilized on the surface of the solid support, e.g. by chemical fixation. Typically, the preferred fixative will depend upon whether the cellular response manifests or the molecule of interest is localized at the cell's surface or within the cell. For example, some fixation methods (such as methanol or acetone fixation) are not usually used on cells that will need to be permeabilized (e.g. examination of intracellular antigens).

Various fixation protocols for various cell types or cell structures for various assays are well known in the art; e.g. mammalian cells may be contacted with a fixative such as phosphate-buffered saline (PBS) with 3.7% para-formaldehyde and 4.0% sucrose.

The term “cellular component” as used in the present invention encompasses any cell types that can be cultured on standard tissue culture ware. Both adherent and non-adherent cell types may be used. A “cellular component” as used in the present invention means any cell or cell structure which allows the detection of a response upon exposure or treatment to/with an effector. A cellular component according to the present specification may be a wild type, a mutant or a transformed or transfected cell (e.g. bacterial cell) and may therefore afford the subsistence or lodgement of a non-host substance; said non-host substance may be viable such as e.g. a parasite or non-viable such as e.g. a vector and may be stably or transiently present in said host cell. A cell has been transfected by exogenous or heterologous genetic material when such material has been introduced inside the cell. A cell has been transformed by exogenous or heterologous genetic material when the transfected material effects a cellular change, e.g. a phenotypic change. The transforming genetic material may be integrated into the cell's chromosomal DNA making up its genome or episomal. Integration of transforming genetic material including vector DNA into the host chromosome may occur by homologous or non-homologous recombination. Episomal includes plasmids either stably replicated or transiently present, or non-integrative viruses and vectors derived thereof. Further, a “cellular component” as used in the present specification encompasses any progeny of a parent cell which is not identical to the parent cell due to mutations that occur during replication.

Useful cells include prokaryotes and eukaryotes such as mammalian cells including hybridoma cells, insect cells, plant cells, yeast cells, and protist cells comprising protozoa, algae and fungal cells. Mammalian cells may be derived from any recognized source with respect to species (e.g. human, rodent, simian), tissue source (brain, liver, lung, heart, kidney, skin, muscle) and cell type (e.g. epithelial, endothelial). In addition, cells which have been transfected with recombinant genes may also be cultured using the present invention. Suitable cell lines may be comprised within e.g. the American Type Culture Collection and the German Collection of Microorganisms and Cell Cultures.

Accordingly, in one embodiment of the present invention, cellular components are selected from the group comprising mammalian cells, insect cells, yeast cells, fungal cells, plant cells, microbial cells, bacterial cells, cellular vesicles, cellular organelles, tissue sections, whole organisms including nematodes.

Non-limiting examples of useful mammalian cell lines include animal and human cell lines such as Chinese hamster ovary (CHO) cells, Chinese hamster lung (CHL) cells, baby hamster kidney (BHK) cells, COS cells, HeLa cells, THP cell lines, Jurkat cells, hybridoma cells, carcinoma cell lines, hepatocytes, primary fibroblasts, endothelial cells, tumour cell lines and the like.

Suitable insect cell lines include but are not limited to Lepidoptera cell lines such as Spodoptera frugiperda cells (e.g. Sf9, Sf21) and Trichoplusia ni cells (e.g. High Five™, BTI-Tn-5B1-4).

Non-limiting examples of fungal cells useful in the present invention include the phyla Ascomycota, Basidiomycota, Chytridiomycota, and Zygomycota as well as the Oomycota and all mitosporic fungi. Representative groups of Ascomycota include, e.g., Neurospora, Eupenicillium (or Penicillium), Emericella (or Aspergillus), Eurotium (or Aspergillus), and the true yeasts listed above. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., saprolegniomycetous aquatic fungi (water molds) such as Achlya. Examples of mitosporic fungi include Aspergillus, Penicillium, Candiada, and Alternaria. Representative groups of Zygomycota include, e.g., Rhizopus and Mucor.

Fungal cells may be yeast cells. Non-limiting examples of useful yeast cells include ascosporogenous yeast (Endomycetales), basidiosporogenous yeast, and yeast belonging to the Fungi Impeffecti or Deuteromycota (Blastomycetes). The ascosporogenous yeasts are divided into the families Spennophthoraceae and Saccharomycetaceae. The latter is comprised of four sub-families, Schizosaccharomycoideae (e.g., genus Schizosaccharomyces including S. pombe), Nadsonioideae, Lipomycoideae, and Saccharomycoideae (e.g., genera Pichia including P. pastoris, P. guillermondii and P. methanolio), Kluyveromyces including K. lactis, K. fragilis and Saccharomyces including S. carlsbergensis, S. cerevisiae, S. diastaticus, S. douglasii, S. kluyveri, S. norbensis or S. oviformis). The basidiosporogenous yeasts include the genera Leucosporidim, Rhodosporidium, Sporidiobolus, Filobasidium, and Filobasidiella. Yeasts belonging to the Fungi Imperfecti are divided into two families, Sporobolomycetaceae (e.g., genera Sporobolomyces and Bullera) and Cryptococcaceae (e.g., genus Candida including C. maltose). Other useful yeast host cells are Hansehula polymorpha, Yarrowia lipolytica, Ustilgo maylis.

Fungal cells may be filamentous fungal cells including all filamentous forms of the subdivision Eumycota and Oomycota. Filamentous fungi are characterized by a vegetative mycelium composed of chitin, cellulose, glucan, chitosan, mannan, and other complex polysaccharides. Vegetative growth is by hyphal elongation and carbon catabolism is obligatory aerobic. In contrast, vegetative growth by yeasts such as Saccharomyces cerevisiae is by budding of a unicellular thallus and carbon catabolism may be fermentative. In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof.

Useful microorganism cells may be unicellular, e.g. a prokaryotes, or non-unicellular, e.g. eukaryotes. Useful unicellular cells are Archeabacteria. Further useful unicellular cells are aerobic bacterial cells such as gram positive bacteria including, but not limited to, the genera Bacillus, Sporolactobacillus, Sporocarcina, Filibacter, Caryophanum, Arthrobacter, Staphylococcus, Planococcus, Micrococcus, Mycobacterium, Nocardia, Rhodococcus; or gram negative bacteria including, but not limited to, the genera Acetobacter, Gluconobacter, Frateuria, Alcaligenes, Achromobacter, Deleya, Amoebobacter, Chromatium, Lamprobacter, Lamprocystis, Thiocapsa, Thiocystis, Thiodictyon, Thiopedia, Thiospirillum, Escherichia, Salmonella, Shigella, Erwinia, Enterobacter, Serratia, Legionella, Neisseria, Kingella, Eikenella, Simonsiella, Alysiella, Nitrobacter, Nitrospina, Nitrococcus, Nitrospira, Pseudomonas, Xanthomonas, Zoogloea, Fraturia, Rhizobium, Bradyrhizobium, Azorhizobium, Sinorhizobium, Rickettsia, Rochalimaea, Ehrlichia, Cowdria, Neorickeftsia, Treponema, Borrelia, Vibrio, Aeromonas, Plesiomonas, Photobacterium, Brucella, Bordetella, Flavobacterium, Francisella, Chromobacterium, Janthinobacterium, and lodobacter.

Suitable plant cells for use in the present invention include dicotyledonous plant cells, examples of which are Arabidopsis Thaliana, tobacco, potato, tomato, and leguminous (e.g. bean, pea, soy, alfalfa) cells. It is, however, contemplated that mono-cotyledoneous plant cells, e.g. monocotyledonous cereal plant cells such as for example rice, rye, barley and wheat, may be equally suitable.

Effector Molecules

Effector molecules relate to any molecule or compound that may affect the cellular components present on the solid support.

Table 1 lists a number of effectors that may be used within the methods of the present invention. In particular, Table 1 summarizes possible combinations of effectors and other reactants that may be supplied from a supply chamber or that may be printed on the solid support at the start of the experiment or analysis. Table 1 shows possible combinations between reactants supplied from a supply chamber with reactants printed on the substrate with other reactants which may be provided or added to the analysis at the start.

Effector molecules may be chosen from the group comprising nutrients, enzyme substrates, test compounds; inducer molecules; chaperone proteins; hormones, oligopeptides including modified analogues thereof; nucleic acids including modified analogues thereof and including synthetic variations thereof such as PNA's or LNA's, agonists; antagonists; inhibitors of cellular functions; enhancers of cellular functions; transcription factors, growth factors; differentiation-inducing agents, secondary metabolites, toxins, glycolipids, carbohydrates, antibiotics, mutagens, drugs; antibodies and antibody fragments including modified analogues thereof, and any combination thereof.

Effectors that may be provided by other means than supply chamber or liquid handling apparatuses include for example electromagnetic treatments, temperature treatment, pressure treatment and the like. Reactants may also be provided during the experiment or analysis. Examples of reactants that may be provided after initiation of the experiment or analysis include for example vital dyes, fixatives, preservatives which may be provided via a supply chamber according to the present invention or alternatively may be sprayed over the cellular array.

In one embodiment of the present invention, methods are provided wherein effector molecules are chosen from the group comprising nutrients, enzyme substrates, test compounds, inducer molecules, chaperone proteins, hormones, oligopeptides, nucleic acids, agonists, antagonists, inhibitors of cellular functions, enhancers of cellular functions, transcription factors, growth factors, differentiation-inducing agents, secondary metabolites, toxins, glycolipids, carbohydrates, antibiotics, mutagens, drugs, proteins, antibodies, antibody fragments, modified analogues thereof, and any combination thereof.

Cellular Responses

The present invention provides a method for screening and/or the pharmacological profiling of test compounds or effectors modulating a cellular response, e.g. a physiological response and/or the activities of cells. A variety of effects caused by the compounds or effectors to be screened may be detected and quantitatively characterized according to the present invention. These effects include but are not limited to changes in intracellular concentration of ionized calcium, CAMP differences (e.g. due to metabolic activation or inactivation), pH, temperature, NO, and trans-membrane potential, intracellular Ca-, K- or Na-fluxes in or out of the cell and other physiological and biochemical characteristics of living cell which can be measured by a variety of conventional means, for example using specific fluorescent, luminescent or colour developing dyes.

The present invention also includes methods of screening for agonist or antagonist activity of drugs, methods of characterizing their potency profiles, methods of identifying the receptor expression pattem of cell membrane (“receptor fingerprinting”), methods of determining toxicity profiles for the compounds (e.g. toxicological responses, CYP450, HERC), bacterial lysis, apoptosis, cellular necrosis, cell mutation processes such as e.g. carcinogenesis, drug induced protein-protein interactions detectable using fluorescence resonance energy transfer (FRET) or bioluminescent resonance energy transfer (BRET), ADME (adsorption, distribution, metabolism and elimination) or any other cellular responses. The plurality of cellular responses includes a cellular response selected from the group consisting of signal transduction, general protein-protein interactions, changes in enzyme activity, vesicle trafficking, protein movement, vesicle movement, activation or inhibition of a receptor mediated response, activation or inhibition of an ion channel, activation or inhibition of a non-selective pore, activation or inhibition of a second messenger pathway at a point downstream of a receptor or channel, activation or inhibition of apoptosis, and activation or inhibition of cellular necrosis, cell behaviour and organism behaviour, cellular toxicity, cell differentiation and cell proliferation, neuroprotection, angiogenesis and alterations of biochemical markers or growth properties as a consequence of recombinant overexpression. Some cellular responses such as bacterial lysis, apoptosis, necrosis, proliferation do not necessarily need detector molecules for them to be detected; instead they may be detected by visual inspection.

The method of the present invention may also be used to perform biochemical analyses, such as Western analyses, Northern analyses, detection of single nucleotide polymorphisms (SNPs), detection of enzymatic activities, or molecular assembly assays.

According to the methods of the present invention, the ability and potency of substances to act as agonists or antagonists against receptors, ion channels, ion pumps, and ion transporters localized on a cell surface membrane may be detected, evaluated and characterized. These molecular assemblies work in concert to maintain intracellular ion homeostasis. Any changes in the activity of these systems would cause a shift in the intracellular concentrations of ions and consequently to the cell metabolic response.

Ion pumps act to maintain trans-membrane ion gradients utilizing ATP as a source of energy. Examples of ion pumps are: ATP synthesis driven by H⁺ gradients, Na⁺/K⁺-ATPase maintaining trans-membrane gradient of sodium and potassium ions, Ca²⁺-ATPase maintaining trans-membrane gradient of calcium ions and H⁺-ATPase maintaining trans-membrane gradient of protons.

Ion transporters use the electrochemical energy of trans-membrane gradients of one ion species to maintain gradients of other ion counterpart. For example, the Na⁺/Ca²+-exchanger uses the chemical potential of the sodium gradient directed inward to pump out calcium ions against their chemical potential.

Ion channels, upon activation, allow for the ions to move across the cell membrane in accordance with their electrochemical potential.

Accordingly, in one embodiment, methods according to the present invention are provided, wherein said cellular responses are chosen from the group comprising chemically induced or physiological events in the cell including lysis, apoptosis, growth inhibition, and growth promotion; morphology changes; cell differentiation; organelle movement; changes in metabolite concentrations or metabolite patterns; changes in cellular contents including changes in mRNA level, protein composition, lipid composition, carbohydrate composition, production of a protein, secretion of a protein, and surface exposure of a protein or other molecule of interest by the cell; membrane surface molecule activation including receptor activation; trans-membrane ion transports; stage of infection to viruses, prions or cellular pathogens or resistance to such pathogens; and cell-cell interactions including changes to communities or mixtures of cells.

In a further embodiment, methods are provided, wherein said molecule of interest is selected from the group comprising peptides including oligopeptides, lipopeptides, glycosylated peptides, antimicrobial peptides, polypeptides, proteins, enzymes, antimicrobial molecules, primary and secondary metabolites, and small organic molecules including pharmaceutical molecules and pharmacophores.

Detection

Cellular responses may be detected in a number of ways. Detection may be by just visual inspection; e.g. cell growth or not, cell morphology, etc. or may be by the use of detector molecules. Detector molecules may be already present in the array of cells; e.g. when looking at expression of a gene with a GFP reporter. Also, the detector molecules may diffuse from the supply chamber into the pores of the porous solid support.

In one embodiment of the present invention detector molecules are selected from the group comprising nucleic acids including modified analogues thereof; peptides and oligopeptides including modified analogues thereof; proteins; antibodies including antibody fragments; aptamers; enzyme substrates; carbohydrates; specific dyes; and combinations thereof.

In one embodiment of the present invention, methods are provided, wherein said detector molecules are present within the pores of the solid support prior to providing cellular components and effectors.

Accordingly in a further embodiment, a device as described herein comprising a porous solid support and a supply chamber is provided, wherein an array of detector molecules is immobilized within said porous support.

The multiplexing character of the invention may be also at the level of the immobilized reactants. For example, detector molecules may be provided within the porous structure of the porous solid support at predefined regions.

Accordingly, in yet a further embodiment, a device as described herein comprising a porous solid support and a supply chamber is provided, wherein an array of detector molecules is immobilized within said porous support and wherein said array of detector molecules comprises a plurality of equal detector molecules or a plurality of different detector molecules.

Where detector molecules are not yet present in the cellular array, cellular responses may be assayed by the addition of the detector molecules to the cellular array after incubation of effectors with cellular components.

Assaying of cellular responses may be by:
(a) providing a detection agent to the cellular components;
(b) optional washing off excess of unincorporated detecting agent; and,
(c) detecting the presence or absence of a change in detectable signal, the presence of a change in detectable signal indicating a cellular response.

Accordingly, in one embodiment of the present invention, methods are provided, wherein said assaying of cellular responses is by: detecting the presence or absence of a change in detectable signal, the presence of a change in detectable signal indicating a cellular response.

Alternatively, label free detection of cellular responses may be envisaged by e.g. calorimetric measurements. This allows the measurement of e.g. metabolic activities in a cell by detection with, for example, a sensitive IR camera.

Detection of cellular responses may be performed directly on the solid support with the cellular components embedded in e.g. the nutrient solution or broth that is supplied via the supply chamber. Alternatively, detection of cellular responses may be performed after a short preparative step. The plate holding the array of arrays may be e.g. centrifuged to allow the cells on the surface of said support to form a pellet that subsequently may undergo a lysis step to expose cell contents for further analysis or detection within the wells of the plate. Alternatively, the supematant may be used for further analysis or detection of cell-released components.

Accordingly, in one embodiment of the present invention, cellular responses are assayed in whole broth or cell culture medium, in isolated cells such as pelleted cells, in supematant of the cellular components, or in lysate of the cellular components.

The present invention contemplates the monitoring of more than one cellular response, by for example looking at fluorescence at different wavelengths by using e.g. CY3 and CY5 dyes, or by simultaneously or sequentially employing different methods for detection.

A number of parameters can be checked in parallel from the top of the array or support while providing the cellular components with effectors from a supply chamber underneath.

Non-limiting examples of parameters that may be monitored during a cell-based assay include enzyme activities, pH and other ion concentrations including gradients across cell membranes that may be detected by indicator dyes requiring for example a fluorescence detector (e.g. microscope). Alternatively, detection may be through radioactivity detected by a phosphor imager or by micro auto-radiography. Reporter genes (classically GFP) could be made sensitive to many environmental conditions or intracellular events.

Detection may be also by use of antibodies or other binding compounds such as lectins. Usually, fluorescence is most commonly used.

Morphology and intracellular organelle movement or structure may be monitored by microscopy and may be aided by interpretive software. Cell viability may be monitored by vital dyes and cell growth by counting cells (including real-time growth kinetics) or by visual inspection for changes in cell structure indicative of stage in the cell cycle. Non-limiting suitable examples of vital dyes are well known in the art and include e.g Fun-1, Fun-2, and the combination of cell permeable and impermeable nucleic acid dyes (see e.g. Molecular Probes catalogue) or dyes that detect membrane potential such at CTC.

Cell interactions may be monitored in a number of ways including for example change in cell morphology and/or growth and/or signaling compounds or by transfer of genetic material indicated by a reporter gene.

Sampling is possible from each compartment of the supply chamber for later assaying by e.g. mass spectroscopy, atomic force microscopy, chemical analysis or genetic analysis.

Sampling may be by e.g. robotic handling with pins or micropippets or by e.g. contact transfer (“blotting”). Samples may be proteins or nucleic acids or other compounds from cells for molecular analysis, e.g. hybridisation or western blotting or other.

Additional parameters that can be monitored from above the support as well as in-situ on-chip (by e.g. ion selective field effect transistors or ISFETS) include gas concentrations such as e.g., oxygen, CO₂, CO, and temperatures (by e.g., IR detectors). These parameters can be indicative for global metabolism of cells or changes therein. The advantage is that no knowledge about molecular pathways is required to measure cellular responses after exposure to effectors.

Cells or cellular components may be modified with luminescent indicators for chemical or molecular cellular properties and may be analysed in a living state.

Said indicators may be introduced into the cells before or after they are challenged with test compounds and by any one or a combination of a variety of physical methods, such as, but not limited to diffusion across the cell membrane, mechanical perturbation of the cell membrane, or genetic engineering so that they are expressed in cells under prescribed conditions. Pre-labelling often implies a covalent attachment of a label. Inside cells this may be accomplished by making a construct with e.g. GFP or a reporter enzyme. Dyes may be introduced in the cells and form a non-covalent complex with e.g. calcium, or change colour upon protonation (luminescent indicators). Some dyes may be used as an indicator in living cells; others may be used to label materials outside the cell. Live studies permit analysis of the physiological state of the cell as reported by the indicator during its life cycle or when contacted with a test compound such as a drug or other reactive substance. A particular useful luminescent indicator as used within the present is a fluorescent indicator.

In one embodiment of the present invention, identifying the cellular responses is through pre-labelling of cellular components by introduction of a luminescent indicator.

Particularly useful fluorescent molecules include, by way of example and not limitation, fluorescein isothiocyanate (FITC), rhodamine, malachite green, Oregon green, Texas Red, Congo red, SybrGreen, phycoerythrin, allophycocyanin, 6-carboxyfluorescein (6-FAM), 2′,7′-dimethoxy4′,5′-dichloro-6-carboxyfluorescein (JOE), 6-carboxy X-rhodamine (ROX), 6-carboxy-2′,4′,7′,4,7-hexachlorofluorescein (HEX), 5-carboxyfluorescein (5-FAM), N,N,N′,N′-tetramethyl-6-carboxyrhodamine (TAMRA), cyanine dyes (e.g. Cy5, Cy3), BODIPY dyes (e.g. BODIPY 630/650, Alexa542, etc), green fluorescent protein (GFP), blue fluorescent protein (BFP), yellow fluorescent protein (YFP), red fluorescent protein (RFP), and the like, (see, e.g., Molecular Probes, Eugene, Oreg., USA now Invitrogen owned).

Dyes may provide useful information either in living cells or in dead cells, e.g. stain specific organelles (e.g. mitochondria) or indicate ion gradients. Yet other dyes may indicate extracellular activities (e.g. secreted enzymes) or cell surface properties (e.g. wheat germ agglutinin conjugated to a fluorescent dye). All these and more are relevant within the present invention.

Fluorescence detection may include for example time resolved fluorescence and fluorescence anisotropy measurements and further also fluorescence lifetime imaging and fluorescence correlation spectroscopy.

Similar to fluorescence, also phosphorescence provides a suitable detection means. Phosphorescence relates to a quasi-stable electron excitation state involving a change of spin state (intersystem crossing) which decays only slowly. It is similar to fluorescence, but the species is excited to a metastable state from which a transition to the initial state is forbidden.

In one embodiment of the present invention, methods are provided, wherein said luminescence is fluorescence or phosphorescence.

Means for detecting signals in general are well known to those of skill in the art. Thus, for example, radiolabels may be detected using photographic film or scintillation counters, fluorescent markers may be detected using a photodetector to detect emitted illumination, enzymatic labels are typically detected by providing the enzyme with an enzyme substrate and detecting the reaction product produced by the action of the enzyme on the substrate, and colorimetric labels are detected by simply visualizing the coloured label. Further detection means are for example (micro) calorimetry and (light)-microscopy.

In one embodiment of the present invention, identifying of the cellular responses is by a method chosen from the group comprising luminescence, regular light microscopy, and electron microscopy.

Detection of cellular responses may also be accomplished by multi-step detection practices. Said practices may be, by way of example and not limitation, sandwich assays as are well-known in the art and enzymatic conversions into a detectable product.

In one embodiment of the present invention, assaying is performed in real-time.

In another embodiment of the present invention, assaying is an end-point assaying

Solid Porous Support

As understood within present specification, the term “first and second surfaces of a support” refers to the outer top and bottom sides of said support. For a porous support, said first and second surfaces may therefore be physically distinct surfaces interconnected by an intermediate material having a plurality of through-going pores or channels or may be an integral part of a porous material.

A number of materials suitable for use as support in the present invention have been described in the art. Materials particularly suitable for use as support in the present invention include any type of porous support known in the art. More materials particularly suitable for use as support in the present invention include any type of solid porous supports known in the art. The term “porous support” as used in the present specification refers to a support possessing or full of pores, wherein the term “pore” refers to a minute opening or microchannel by which matter may be either absorbed or passed through. Particularly, where the pores allow passing-through of matter, the support is likely to be permeable.

It is understood that porous supports according to the present invention may be semi porous. Semi porous supports can be induced to become fully porous by e.g. a chemical treatment or an illumination treatment. The use of semi porous supports is advantageous in particular if the mixing of (short living) components within the supply chamber compartment(s) and/or within the pores of the porous support in a synchronous manner at a certain time in an assay is envisaged or required.

The support may be in the form of porous beads, particles, sheets, films or membranes. For example, the support may consist of fibres (such as glass wool or other glass or plastic fibres), glass or plastic capillary tubes, or metal oxide membranes. The porous support may have simple or complex shape. The surface to which the molecule is adhered may be an external surface or an internal surface of the porous support. Particularly where the support material is porous, the molecule is likely to be attached to an internal surface. Where the solid support is porous, various pore sizes may be employed depending upon the nature of the system.

The material of the porous support may be, for example, a metal, a ceramic metal oxide or an organic polymer. As a metal, for example, a porous support of stainless steel (sintered metal) may be used. For applications not requiring heat resistance, a porous support of an organic polymer may also be used. Above all, in view of heat resistance and chemical resistance, a metal oxide may be used. In addition, metal oxides provide a support having both a high channel density and a high porosity, allowing high density arrays comprising different target molecules per unit of the surface for sample application. In addition, metal oxides are highly transparent for visible light. Metal oxide supports are relatively cheap and do not require the use of any typical microfabrication technology and, that offer an improved control over the liquid distribution over the surface of the substrate, such as electrochemically manufactured metal oxide membrane. Metal oxide membranes having through-going, oriented channels may be manufactured through electrochemical etching of a metal sheet.

According to one embodiment of the present invention, methods are provided wherein said solid support is a metal oxide solid support.

The kind of metal oxide is not especially limited. Metal oxides considered are, among others, oxides of zirconium, mullite, cordierite, titanium, zeolite or zeolite analog, tantalum, and aluminium, as well as alloys of two or more metal oxides and doped metal oxides and alloys containing metal oxides.

Accordingly, in a further embodiment of the present invention, methods are provided wherein said metal oxide solid support is an aluminium oxide solid support. Metal oxide supports or membranes as employed in the methods of the present invention may be anodic oxide films. As well known in the art, aluminium metal may be anodized in an electrolyte to produce an anodic oxide film. The anodization process results in a system of larger pores extending from one face and interconnects with a system of smaller pores extending in from the other face. Pore size is determined by the minimum diameters of the smaller pores, while flow rates are determined largely by the length of the smaller pores, which can be made very short. Accordingly, such membranes may have oriented through-going partially branched channels with well-controlled diameter and useful chemical surface properties. Useful thicknesses of the metal oxide supports or membranes as employed in the methods and apparatuses of the present invention may for instance range from 50 μm to 150 μm (including thicknesses of 60, 70, 80, 90, 100, 110,120, 130 and 140 μm). A particular suitable example of substrate thickness is 60 μm. A suitable substrate pore diameter ranges from 150 to 250 nm including 160, 170, 180, 190, 200, 210, 220, 230 and 240 nm. A particular suitable example of pore diameter is 200 nm. These dimensions are not to be construed as limiting the present invention.

Due to the characteristic porous structure of the solid supports according to the present invention minimal amounts of reactants or compounds may be deposited on its surface or within the pores but be accessible to cells at an effective concentration; e.g. an antifungal antibiotic that is active at concentrations below 1 microgram per millilitre can be printed on the surface of the porous solid support at 100 picograms per square millimetre and guarantee killing and inhibition of germ tube growth by a fungal pathogen. Accordingly, only picogram quantities of a drug may be required to give a local concentration (to cellular components on the surface) of about 1 microgram per ml.

Accordingly, the solid supports according to the present invention offer advantages in terms of minimal amounts of printed compound having an effect. This may be due to the pore structure of the solid support trapping compounds in close proximity to cellular components.

Advantageously, metal oxide membranes as described herein are transparent, especially if wet, which allows for assays using various optical techniques. WO 99/02266 which discloses the Anopore™ porous membrane or support is exemplary in this respect, and is specifically incorporated by reference in the present invention.

Particular useful porous supports as employed in the methods described in the present specification are 3-dimensional supports, which allow pressurized movement of fluid, e.g. the sample solution, through its structure. As such, particular useful porous supports as employed in the present methods possess a permeable or flow-through nature. In contrast with two-dimensional supports, 3-dimensional supports or microarrays as employed in the methods as described herein give significantly reduced hybridisation or reaction times and increased signal and signal-to-noise ratios. Further, a positive or negative pressure may be applied to the arrays in order to pump the sample solution dynamically up and down through the support pores. Said dynamical pumping allows immediate removal and ability to perform real-time detection of generated products from a reaction which takes place within the pores of the support by fast binding of said generated products to the substrate pore walls or on or within the cells on the surface.

Accordingly, in one embodiment of the present invention, methods are provided wherein said solid support is a flow-through solid support.

The nature and geometry of the porous support as useful in the present invention will depend upon a variety of factors, including, among others, the type of array and the mode of attachment of effectors and even cellular components (e.g., covalent or non-covalent). Generally, the substrate according to the present invention may be composed of any porous material which will permit immobilization of a probe-molecule and which will not melt or otherwise substantially degrade under the reaction and incubation and detection conditions used.

Applications

The methods and devices according to the present invention are useful in ample applications.

In one embodiment, the present invention provides for the use of methods as described herein for monitoring induced cellular responses of host cells.

In one embodiment, the present invention provides for the use of methods as described herein for monitoring real-time growth kinetics on-chip.

In one embodiment, the present invention provides for the use of methods as described herein for monitoring cell morphology.

In one embodiment, the present invention provides for the use of methods as described herein for monitoring cell behaviour.

In one embodiment, the present invention provides for the use of methods as described herein for monitoring sub-cellular vesicle trafficking.

In one embodiment, the present invention provides for the use of methods as described herein for on-chip recombination, transformation or viral introduction of cellular components

In one embodiment, the present invention provides for the use of methods as described herein for functional screening of cellular responses upon assaying host cells or organisms with test compounds.

In one embodiment, the present invention provides for the use of methods as described herein for biofilm modelling.

In one embodiment, the present invention provides for the use of a device as described herein for cell-based assays according to a method as described in any of claims 1 to 28.

In one embodiment, the present invention provides for the use of a device as described herein for applications as defined in any of claims 30 to 37.

It is a further object of the present invention to provide a kit for performing a method as provided by the present invention, comprising a device as provided by the present invention.

### SHORT DESCRIPTION OF THE FIGURES

The following Figures of the invention are exemplary and should not be taken as in any way limiting.

FIG. 1A illustrates a device according to the present invention comprising a supply chamber (SC) and a porous solid support. The porous solid support is present at the bottom of each well in a plate or carrier comprising an array of wells to form an array of arrays (AA). The design in this figure shows a compartmentalized supply chamber comprising a multitude of square-shaped compartments (c) that is placed underneath the solid support and wherein each compartment of the supply chamber covers a number of arrays (a).

FIG. 1B illustrates a device similar as shown in FIG. 1A wherein the supply chamber comprises compartments with different contents that supply certain content (e.g. nutrients (1)) only to a limited number of corresponding arrays in the array of arrays. The compartmentalization may be so that there is a 1:1 correspondence to an array (2).

FIG. 2 illustrates the holder and chips as used in the experiments as described in the Example.

FIG. 2A: FD10 disposable used as a holder contains a laminated porous support exposing four test areas;

FIG. 2B shows the four test area laminated porous support. 1, upper surface of test area where bacteria were inoculated and grown; 2, plastic laminate; 3, FD10 disposable housing.

FIG. 3 illustrates the supply of nutrients through the porous support from underneath to bacteria on the outer top surface of said porous support by a hanging drop of nutrient medium. The top panel of FIG. 3 is a view from above the porous support showing a mass of fluorescent bacteria on the surface. The bottom panel of FIG. 3 is a schematic view from the side of the porous support.

A, porous support; B, bacteria on upper surface of the porous support; C, hanging drop of nutrient medium under the test area supplying the bacteria with nutrients; a, view from above; b, view from the side.

FIG. 4 illustrates the bacterial growth assay using a supply chamber as described in the Example (see 2, “Supply of nutrients via a supply chamber”). Experiment was carried out with E coli.

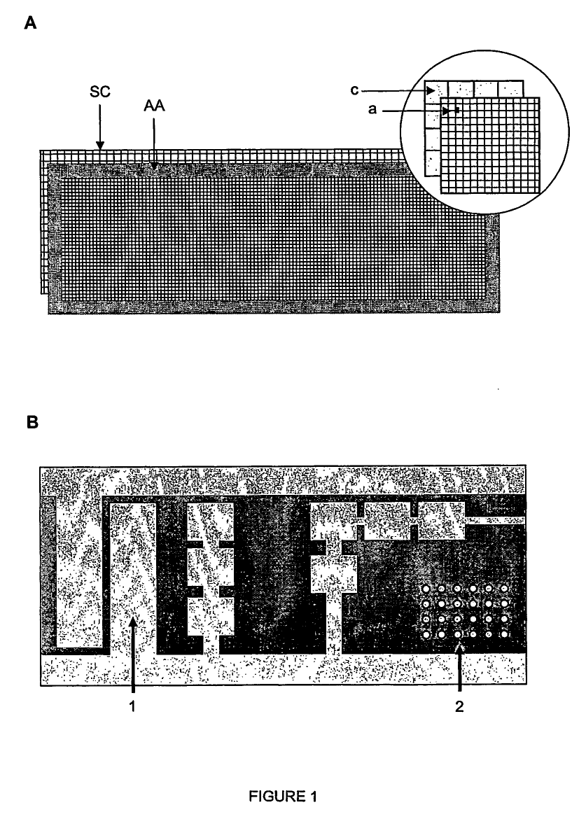

The scale bar indicates 0.8 mm for A-D, and 10 μM for E and F. FIG. 5 illustrates the bacterial growth assay using a supply chamber as described in the Example (see 2, “Supply of nutrients via a supply chamber”). Experiment was carried out with a mixture of E. coli and S. aureus. Scale bar in B represents 0.8 mm for A and B, and 5 μM for C.

### EXAMPLE

Growth of Bacteria on a Porous Support by which Nutrients are Supplied from Underneath

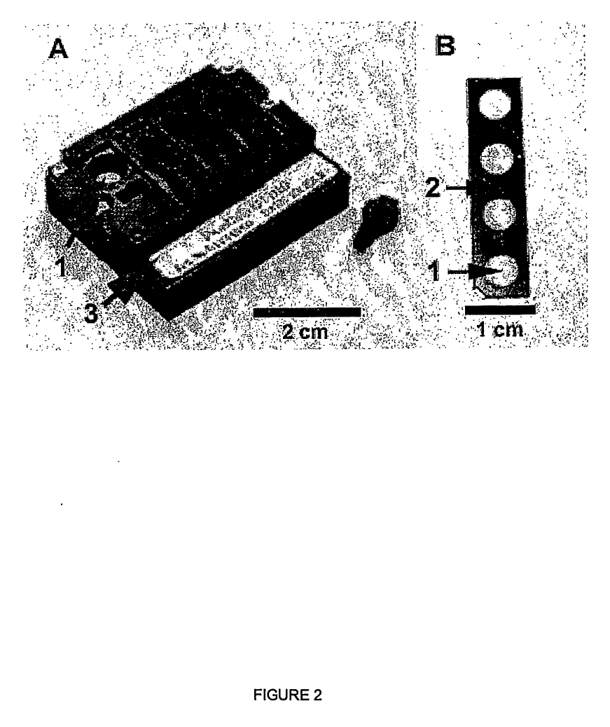

As shown in FIG. 2, a simple set up comprising a supply chamber and four test areas was used to demonstrate that cells can be grown an assayed on the top surface of a porous support when supplied with nutrients in liquid form from underneath. A strip of 36×8 mm porous aluminium oxide (Anopore™) was laminated in a plastic film having 4 open areas so that four test areas of the porous aluminium oxide strip of approximately 4 mm in diameter were exposed. These so-called chips were ethanol sterilized and placed in a plastic disposable holder (FD10; PamGene B. V.) which had also been ethanol sterilized.

In some experiments a filter-sterilized antibiotic (rifampicin dissolved in DMSO at 500 ng/microlitre) was spotted onto one or more of the test areas and air dried so that the rifampicin coated the pores of the porous support.

In the present example, the growth of bacteria on the porous support was studied by which nutrients were supplied from underneath by a hanging-drop in a supply chamber.

1. Experimental Set Up

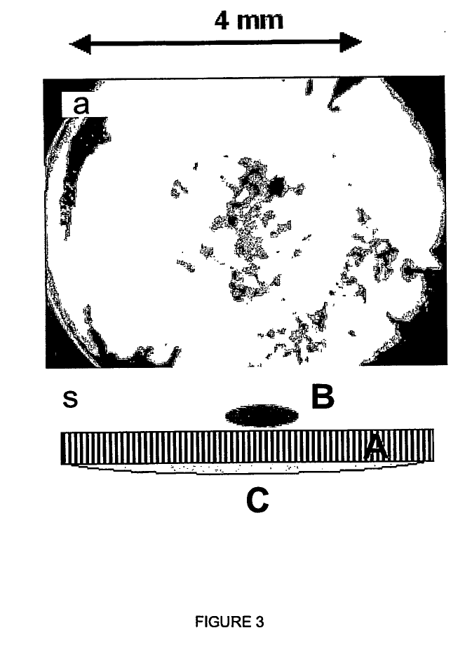

Aliquots (20 μl) of sterile L-broth containing either no bacteria or 200 colony forming units (cfu) of one or both of Staphylococcus aureus (strain 111017) and/or Escherichia coli (strain XL2 Blue) were pipetted into each test area. Both bacterial strains were rifampicin-sensitive. The L-broth was drawn through the pores of the porous support (by suction from below using a 20 ml syringe) so that the bacteria were pulled onto the outer top surface of the porous support but were unable to penetrate it due to the small pore size of the support material. The upper surface of the test area was just barely wet but not flooded with growth medium. The bulk of the L-broth formed a hanging drop under the porous support as well as filling the pores—the net result is that the bacteria are supplied with nutrients through the porous support (FIG. 3b), this is similar to the supply by a supply chamber filled with nutrient solution contacting the outer bottom surface of the porous support Incubation was for 2-3 hours at 37° C. in a humidity chamber. The fluorescent dye Syto9 (Invitrogen) was then pipetted onto the upper surface of the porous support and the bacteria visualised by fluorescence microscopy. Data was captured with a 12-bit Kappa CCD camera controlled by Kappa ImageBase software. Alternatively, FISH oligonucleotide probes were used for detection of bacterial rRNA where noted. In all cases, where staining required fluid to be pulled through the porous support, it was done from the upper surface (where the cells are) to the lower so that the cells were not removed from the chamber.

2. Supply of Nutrients Via a Hanging Drop in a Supply Chamber

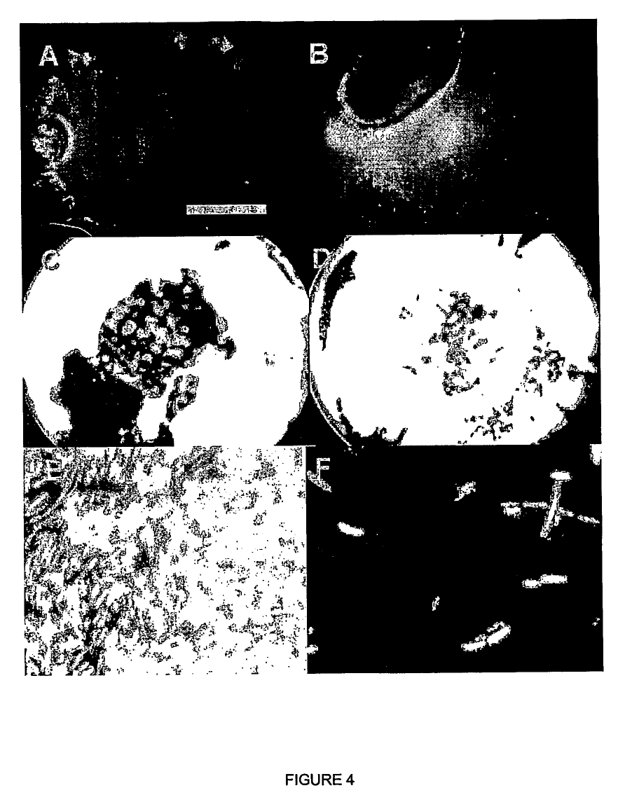

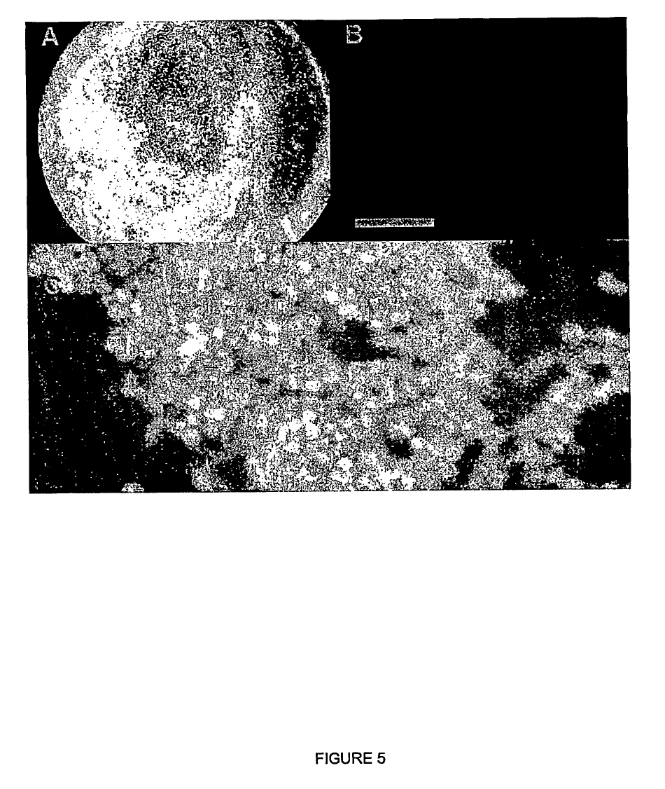

FIG. 4 illustrates a growth assay using a supply chamber to supply nutrient medium from underneath the porous support.

E. coli was inoculated into duplicate test areas A and B previously printed with 200 ng of the antibiotic rifampicin (FIGS. 4A and 4B), or into duplicate test areas C and D with no antibiotics (FIGS. 4C and 4D). After 3 hours growth the test areas A to D were stained using Syto9 (5 μl of a 30 μM stock solution). Test areas were imaged directly on the porous support by a low powered objective lens (×4 Plan) using the appropriate filters. Inhibition of growth was obvious in test areas A and B compared to the growth of bacteria observed in test areas C and D. To check that the fluorescence observed was genuinely due to bacterial growth, test area C was imaged at a sufficiently high magnification (×50 UmPlan F1 objective) to observe cell morphology (FIG. 4E) and a dense aggregate of bacteria was observed as expected. Similarly, high-powered imaging of test area A is shown in FIG. 4F, here the bacterial density was low confirming the effectiveness of the antibiotic.

FIG. 5 also illustrates a growth assay using a supply chamber. Here, a mixture of E. coli and S. aureus were inoculated in two test areas: area A with no antibiotics (FIG. 5A), and area B with 200 ng rifampicin (FIG. 5B). After 3 hours growth, the bacteria in the test areas were ethanol fixed and treated with a mixture of two FISH probes complementary to rRNA sequences. Fish probe F1 was end-labelled with Cy3 and hybridised to all Eubacterial rRNA sequences. F1 will detect both cell types. Fish probe F2 was end-labelled with Cy5 and was specific to S. aureus. The net effect is to label E. coil by hybridization of probe F1 to its rRNA and S. aureus with both F1 and F2. Test areas were then imaged directly on the porous support by a low powered objective lens (×4 Plan) using fluorescence microscopy. Inhibition of growth was obvious in test area B, compared to the growth of bacteria observed in test area A. To check that the fluorescence probes correctly targeted the appropriate species, test area A was imaged at a high magnification (×50 UmPlan F1 objective) to observe cell morphology (shown in FIG. 5C). Yellow cocci and blue rods were observed, as expected.

TABLE 1
Summary of reactants that may be supplied from a supply chamber or that are printed on
the solid support at the start of an experiment or analysis. Some analysis may require
an additional provision of other reactants that are not yet provided at the start and that
may be added during the experiment or analysis. It is noted that all listed reactants may
be provided in all possible combinations, either simultaneously or sequential.

| From supply chamber | Printed on support | Other |
|---|---|---|
| Nutrients | Drugs or antibiotics or adjuncts to | Cellular component (or mixtures |
| | drug or antibiotics action (inhibitors, | thereof) |
| | cofactors) | |
| Buffers (osmotic or pH) | Nutrient supplements (e.g. individual | Preservatives, fixatives, agents to |
| | vitamins or amino acids) | stop enzymatic reactions, fluorescent |
| | | quenching agents |
| Enzyme substrates | Lyophilised cells | Vital dyes, tracers (e.g. radioactivity) |
| | | and other detection agents for cellular |
| | | function or visualization |
| Drugs or antibiotics | Natural products or derivatives | Light, radiation, other electromagnetic |
| | thereof | agents or temperature changes |
| Secreted compounds from | Proteins, nucleic acid, | Effectors, inducers |
| other organisms, e.g. | carbohydrates, and other | |
| hormones | macromolecules both natural and | |
| | synthetic analogues | |
| Vital dyes, tracers (e.g. | Scaffolds (complex protein or | Agonists, antagonists |
| radioactivity) and other | carbohydrate structures or synthetic | |
| detection agents for cellular | analogues) that affect cell growth or | |
| function or visualization | differentiation | |
| Viscous agents to regulate | Agents affecting adhesion of cells | Proteins, antibodies, antibody |
| the rate of fluid entry into the | (lectins, polylysine, antibodies, anti- | fragments, aptamers |
| substrate (e.g. glycerol) | adhesion agents) | |
| Detergents | Slow release agents for other | Toxins, mutagens |
| | compounds printed on the substrate | |
| Toxins, infectious agents, | Fluorescence to enable focusing | Lysing agents, washing liquids |
| mutagens | and monitoring of release of | |
| | compounds printed on the substrate. | |
| Preservatives, fixatives, | Toxins, infectious agents, mutagens | vectors, transfection agents |
| agents to stop enzymatic | | |
| reactions, fluorescent | | |
| quenching agents | | |
| Fractions from a fractionated | Fractions from a fractionated | |
| complex mixture | complex mixture | |
| Inducers (of gene expression, | Inducers (of gene expression, | |
| cellular processes, | cellular processes, pathologies, | |
| pathologies, differentiation) | differentiation) | |
| Proteins, nucleic acid, | | |
| carbohydrates, and other | | |
| macromolecules both natural | | |
| and synthetic analogues | | |
| Transfection reagents | | |
| Transcription factors | | |
| Agonists Antagonists | | |

1. A method for screening of cellular responses comprising:
(a) providing a solid porous support having first and second surfaces and at least one area with a plurality of through-going channels;
(b) providing cellular components on said first and/or second surface of said solid porous support, wherein said solid porous support retains said cellular compounds on its surface;
(c) providing a supply chamber at said first and/or second surface and opposite to said cellular components;
(d) subjecting all or part of said cellular components to one or more effectors; wherein at least one effector is delivered from said supply chamber through the porous support;
(e) incubating the said all or part of cellular components with said effectors under conditions allowing the induction of cellular responses in the said all or part of cellular components;
(f) optionally providing detector molecules to the said all or part of cellular components for assaying cellular responses;
(g) assaying for cellular responses; and
(h) identifying and characterizing the cellular responses induced by said effector molecules.

2. The method according to claim 1 wherein said supply chamber comprises at least one compartment.

3. The method according to claim 2, wherein said at least one compartment is provided with a liquid medium comprising at least one effector molecule.

4. The method according to claim 2, wherein said at least one compartment is provided with a liquid medium comprising a gradient of at least one effector molecule.

5. The method according to claim 2, wherein said at least one compartment is provided with a liquid medium comprising a 2D gradient of at least two effector molecules.

6. The method according to claim 1, wherein said effector molecules are chosen from the group comprising nutrients, enzyme substrates, test compounds, inducer molecules, chaperone proteins, hormones, oligopeptides, nucleic acids, agonists, antagonists, inhibitors of cellular functions, enhancers of cellular functions, transcription factors, growth factors, differentiation-inducing agents, secondary metabolites, toxins, glycolipids, carbohydrates, antibiotics, mutagens, drugs, proteins, antibodies, antibody fragments, modified analogues thereof, and any combination thereof.

7. The method according to claim 1, wherein said supply chamber is in liquid contact with said first and/or said second surface of said solid support.

8. The method according to claim 1, wherein the said at least one effector molecule is transported passively or actively through said porous support.

9. The method according to claim 1, wherein the said at least one effector molecule diffuses through said porous support to the cellular components by contact force.

10. The method according to claim 1, wherein the said at least one effector molecule is transported actively through said porous support by pumping, magnetically, electrically, or by piezo-electronic force.

11. The method according to claim 1, wherein said providing of cellular components on the surface of a support is by a deposit directly on said support of an inoculum, culture, solution, or mixtures thereof.

12. The method according to claim 1, wherein said cellular components are selected from the group comprising mammalian cells, insect cells, yeast cells, fungal cells, plant cells, microbial cells, bacterial cells, cellular vesicles, cellular organelles, tissue sections, whole organisms and nematodes.

13. The method according to claim 1, wherein said detector molecules are selected from the group comprising nucleic acids, modified nucleic acid analogues, peptides, modified peptide analogues, oligopeptides, modified oligopeptide analogues, proteins, antibodies, antibody fragments, aptamers, enzyme substrates, carbohydrates, specific dyes, and combinations thereof.

14. The method according to claim 1, wherein said cellular responses are chosen from the group comprising chemically induced or physiological events in the cell, lysis, apoptosis, growth inhibition, growth promotion, morphology changes, cell differentiation, organelle movement, changes in metabolite concentrations or metabolite patterns, changes in cellular contents; changes in mRNA level, protein composition, lipid composition, carbohydrate composition, production of a protein, secretion of a protein, surface exposure of a protein, or other molecule of interest by the cell; membrane surface molecule activation, receptor activation, trans-membrane ion transports; stage of infection to viruses, prions or cellular pathogens or resistance to such pathogens; cell-cell interactions, and changes to communities or mixtures of cells.

15. The method according to claim 14, wherein said molecule of interest is selected from the group comprising peptides, oligopeptides, lipopeptides, glycosylated peptides, antimicrobial peptides, polypeptides, proteins, enzymes, antimicrobial molecules, primary and secondary metabolites, small organic molecules, pharmaceutical molecules and pharmacophores.

16. The method according to claim 6, wherein said effector is a drug or any compound which is useful in the discovery process of a drug candidate.

17. The method according to claim 16, wherein said effector is a drug selected from a chemical or natural drug candidate library.

18. The method according to claim 1, wherein said cellular response is assayed in whole broth or cell culture medium, in isolated cells, pelleted cells, in supernatant of the cellular components, or in lysate of the cellular components.

19. The method according to claim 1, wherein said assaying of cellular responses is by detecting the presence or absence of a change in detectable signal, the presence of a change in detectable signal indicating a cellular response.

20. The method according to claim 1, wherein delivery of at least one effector is from above the support by a means chosen from the group comprising a delivery mask, a microfluidics device, a high precision x-y-z micro-pipettor, inkjet printer, and manual handling.

21. The method according to claim 1, wherein said identifying of the cellular responses is by a method chosen from the group comprising luminescence, regular light microscopy, and electron microscopy.

22. The method according to claim 21, wherein said luminescence is fluorescence or phosphorescence.

23. The method according to claim 1, wherein said solid support is a flow through solid support.

24. The method according to claim 1, wherein said solid support is a metal oxide solid support.

25. The method according to claim 24, wherein said metal oxide solid support is an aluminium oxide solid support.

26. The method according to claim 1, wherein said assaying is performed in real-time.

27. The method according to claim 1, wherein said assaying is an end-point assaying.

28. The method according to claim 1, wherein said cellular components are pre-labelled by introduction of a luminescent indicator.

29. The method according to claim 1, wherein said detector molecules are present within the pores of the solid support prior to providing cellular components and effecters.

30-37. (canceled)

38. A device for performing a method according to claim 1, comprising a solid porous support; said support being at its first and/or second surface in liquid contact with a supply chamber or in gaseous contact or wherein said supply chamber may be physically attached thereto; wherein said supply chamber comprises multiple use insertions, said multiple-use insertions are fixed or movable separations and wherein the spatial organization of the inserts determines the number of compartments.

39. The device according to claim 38, wherein said supply chamber comprises at least one compartment.

40. The device according to claim 38, wherein an array of test compounds is provided within predefined regions on the surface of said support, said test compounds are in solid, liquid, gaseous or supercritical state.

41. The device according to claim 38, wherein an array of cellular components is provided in predefined regions on the surface of said support.

42. The device according to claim 41, wherein said cellular components are conditioned for preservation on said support.

43. The device according to claim 38, wherein a cellular component is provided on the surface of said support.

44. The device according to claim 43, wherein a cellular component is provided on the surface of said support, said cellular component being conditioned for preservation on said support.

45. The device according to claim 38, wherein an array of detector molecules is immobilized within said porous support.

46. The device according to claim 45, wherein said array of detector molecules comprises a plurality of equal detector molecules or a plurality of different detector molecules.

47. The device according to claim 42, wherein said condition is chosen from the group comprising lyophilization, liquid nitrogen and glycerol dissolution.

48. A solid porous support, wherein within its porous structure an array of chemical compounds is provided in dried, lyophilized, gaseous or supercritical state.

49. A supply chamber for spatial delivery of one or more effectors through a porous solid support comprising:
(a) multiple-use insertions, said multiple-use-insertions are fixed or movable separations and wherein the spatial organization of the inserts determines the number of compartments, said supply chamber comprising at least one compartment, said at least one compartment allowing said delivery of one or more effectors through part or all of the channels within said porous solid support;
(b) means for compartment alignment towards predefined regions on the support;
(c) means of adding or removing or changing the amounts of effectors.

50. The supply chamber according to claim 49, wherein said at least one compartment is provided with one or more effectors.

51. The supply chamber according to claim 50, wherein said at least one or more effectors is contained within a gaseous or liquid medium.

52-54. (canceled)
